(19) United States
(12) Patent Application Publication
(10) Pub. No.: US 2022/0379012 A1
(43) Pub. Date: Dec. 1, 2022

(54) INFUSION DEVICE AND METHOD WITH USER INTERFACE

(71) Applicant: Fresenius Vial SAS, Brézins (FR)

(72) Inventors: Julie Lambert, Grenoble (FR); Cyril Deleaval, L'Albenc (FR); Olivier Carrobourg, Cessieu (FR)

(21) Appl. No.: 17/737,956

(22) Filed: May 5, 2022

(30) Foreign Application Priority Data
May 27, 2021 (EP) 21315091.5

(51) Int. Cl. A61M 5/142; A61M 5/172; A61M 5/168

(52) U.S. Cl. CPC A61M 5/14244; A61M 5/172; A61M 5/16831; A61M 2205/18

(57) ABSTRACT

An infusion device includes a pump configured to deliver a fluid in at least a therapy delivery mode and a stand-by delivery mode. The infusion device includes an audible alarm and at least first, second and third sets of visible indicators. The infusion device includes a pump controller coupled to the pump, the audible alarm, and the visible indicators. The pump controller is configured to operate the pump in the therapy delivery mode or the stand-by delivery mode, and to actuate the first set of visible indicators in the therapy delivery mode and the stand-by delivery mode, to actuate the second set and the audible alarm at an end of the therapy delivery mode and at intervals during the stand-by delivery mode, and to actuate the third set during the stand-by delivery mode when the second set is deactivated.

The present application claims priority to EP Application No. 21315091.5, filed May 27, 2021, which application is hereby incorporated herein by reference.

### TECHNICAL FIELD

The present disclosure is generally directed to infusion devices and methods. More particularly, the present disclosure is directed to infusion devices and methods incorporating a user interface for displaying information regarding infusion processes carried out by the infusion devices.

### BACKGROUND

Automated infusion devices are a commonplace feature of modern healthcare. These devices can be programmed to deliver medical fluids, such as medications, at one or more delivery rates from a container, such as a syringe or a flexible bag, through a delivery line. The delivery line may be connected to a patient via an intravenous needle, for example.

As one such example, an infusion device may be programmed to deliver fluid from the container to a patient at a therapeutic rate. Once a particular delivery volume has been achieved, the infusion device may be programmed to deliver fluid from the container to a patient at a second, continuous rate so as to maintain the vein associated with the needle open for future use. This second rate may be referred to as the “keep vein open”, or KVO, rate, and the associated operational mode as the KVO mode or state.

While the infusion device may be programmed to operate automatically with minimal or no human intervention, and to change automatically between different modes with different delivery rates, it may be useful to communicate this information to the user of the infusion device, which user may be a healthcare professional for example. It would be advantageous for the information to be conveyed via a user interface in a form that is easy to receive. Additionally, it would be advantageous for the information to be conveyed in form that is consistent, such that similar information is communicated in the same or similar forms and that different information is communicated in separate or distinct forms. Further, it would be advantageous that the information regarding changes in the operation of the infusion device be reinforced by using more significant changes in the manner in which the information is communicated to the user.

### SUMMARY

In a first aspect, an infusion device includes a pump configured to be connected to a container and to deliver a fluid from the container during at least a therapy delivery mode and a stand-by delivery mode. The infusion device also includes an audible alarm and a plurality of visible indicators, the plurality of visible indicators including at least a first set of visible indicators, a second set of visible indicators, and a third set of visible indicators. The infusion device includes a pump controller coupled to the pump, the audible alarm, and the plurality of visible indicators, the pump controller configured to operate the pump in the therapy delivery mode or the stand-by delivery mode, and to actuate the audible alarm and the plurality of visible indicators. The pump controller is configured to actuate the first set of visible indicators in the therapy delivery mode and the stand-by delivery mode, to actuate the second set of visible indicators and the audible alarm at an end of the therapy delivery mode and at intervals during the stand-by delivery mode, and to actuate the third set of visible indicators during the stand-by delivery mode when the second set of visible indicators is deactivated.

In a second aspect, an infusion device includes a pump configured to be connected to a container and to deliver a fluid from the container in at least a therapy delivery mode and a stand-by delivery mode. The infusion device includes an audible alarm and a plurality of visible indicators, the plurality of visible indicators including at least a first set of visible indicators, a second set of visible indicators, and a third set of visible indicators. The infusion device includes a pump controller coupled to the pump, the audible alarm, and the plurality of visible indicators. The pump controller is configured to operate the pump in a therapy delivery mode to deliver fluid from the container and actuate the first set of visible indicators, end the therapy delivery mode and actuate the audible alarm and the first and second sets of visible indicators, operate the pump in a stand-by delivery mode to deliver fluid from the container and actuate the first and third sets of visible indicators, and at intervals during operation of the pump in the stand-by delivery mode, deactivate the third set of visible indicators and actuate the audible alarm and the second set of visible indicators.

In a third aspect, a method for operating an infusion device is provided The infusion device includes a pump configured to be connected to a container, an audible alarm, and a plurality of visible indicators, the plurality of visible indicators including at least a first set of visible indicators, a second set of visible indicators, and a third set of visible indicators. The method includes operating the pump in a therapy delivery mode to deliver fluid from the container during which the first set of visible indicators is automatically actuated, ending the therapy delivery mode and automatically actuating the audible alarm and the first and second sets of visible indicators, operating the pump in a stand-by delivery mode to deliver fluid from the container during which the first and third sets of visible indicators are automatically actuated, and at intervals during operation of the pump in the stand-by delivery mode, automatically deactivating the third set of visible indicators and automatically actuating the audible alarm and the second set of visible indicators.

### DESCRIPTION OF THE DRAWINGS

FIG. 1 is a view of an embodiment of an infusion device including a syringe pump, schematically illustrating a pump controller coupled to the syringe pump and the pump configured to deliver fluid from a container to a patient.

FIG. 2 is an enlarged, fragmentary perspective view of the embodiment of the infusion device of FIG. 1.

FIG. 3 is a flow chart of a method of operating an infusion device according to the embodiment of FIG. 1.

FIG. 4 is a side view of an embodiment of the infusion device including a volumetric (peristaltic) pump.

### DETAILED DESCRIPTION

A detailed description of an infusion devices and methods in accordance with the present disclosure is set forth below. It should be understood that the description below of specific devices and methods is intended to be exemplary, and not exhaustive of all possible variations or applications. Thus, the scope of the disclosure is not intended to be limiting, and should be understood to encompass all variations or embodiments that would occur to persons of ordinary skill.

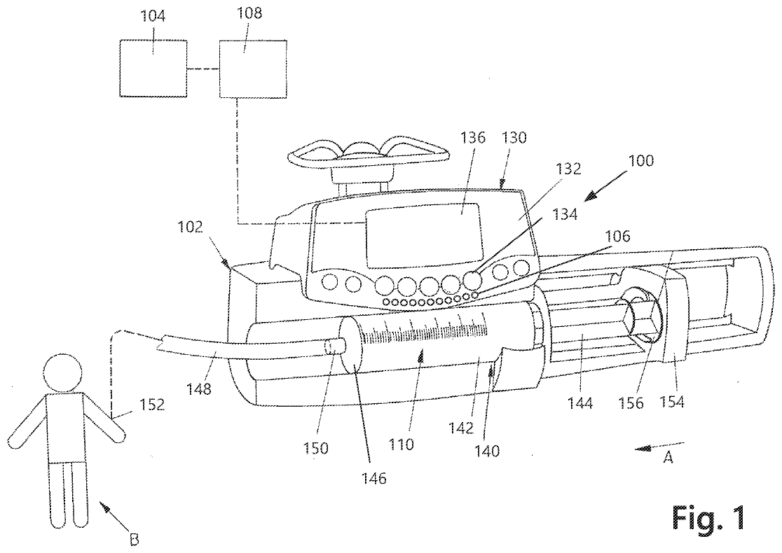

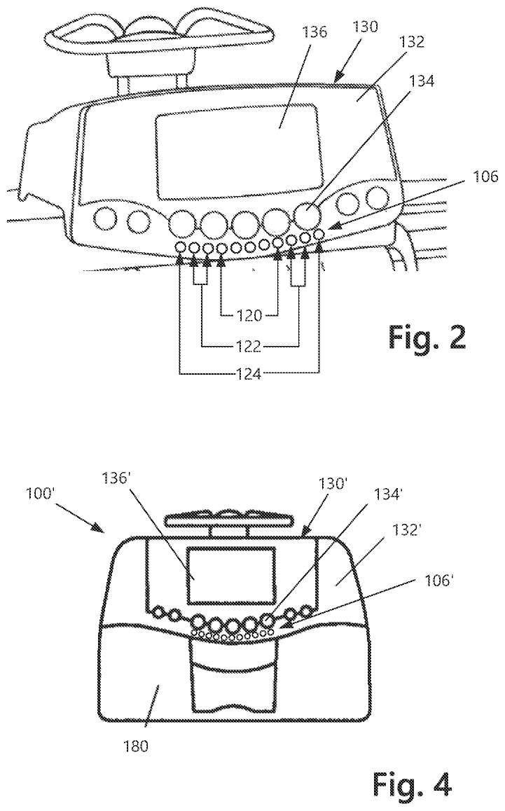

Before turning to specifics of the infusion devices and methods incorporating or including a user interface according to the present description, the general concepts of such an infusion device 100 and method are described with reference to FIGS. 1 and 2. As discussed herein, the infusion device 100 is capable of at least two delivery modes, or states, wherein the infusion device 100 delivers fluid from a container. As illustrated in FIG. 1, the fluid may be delivered from the container to a patient B. The delivery modes may include a (first) therapy delivery mode, wherein the fluid is delivered to the patient from a container at a therapeutic rate or rates. The delivery modes may also include a (second) stand-by mode, wherein fluid is delivered to the patient from a container at a continuous rate to keep a vein to which an infusion line is connected open for future use (a KVO (“keep vein open”) rate).

As illustrated in FIG. 1, the infusion device 100 includes a pump 102, an audible alarm 104, a plurality of visible indicators 106, and a pump controller 108. The pump 102 is configured to be connected to a container 110 and to deliver a fluid from the container 110 during at least the therapy delivery mode and the stand-by (KVO) delivery mode. The pump controller 108 is coupled (e.g., hardwired) to the pump 102, the audible alarm 104, and the plurality of visible indicators 106. The pump controller 108 is configured to operate the pump 102 in the therapy delivery mode or the stand-by delivery mode, and to actuate the audible alarm 104 and the plurality of visible indicators 106.

As illustrated in FIG. 1, and in greater detail in FIG. 2, the plurality of visible indicators 106 includes at least a first set of visible indicators 120, a second set of visible indicators 122, and a third set of visible indicators 124. The pump controller 108 is configured to actuate the first set of visible indicators 120 in the therapy delivery mode and the stand-by delivery mode, to actuate the second set of visible indicators 122 and the audible alarm 104 at an end of the therapy delivery mode and at intervals during the stand-by delivery mode, and to actuate the third set of visible indicators 124 during the stand-by delivery mode when the second set of visible indicators 122 is deactivated.

Stated slightly differently, the pump controller 108 may be configured to operate the pump 102 in a therapy delivery mode to deliver fluid from the container 110 and actuate the first set of visible indicators 120. Further, the controller 108 may be configured to end the therapy delivery mode and actuate the audible alarm 104 and the first and second sets of visible indicators 120, 122. Moreover, the controller 108 may be configured to operate the pump in a stand-by delivery mode to deliver fluid from the container 110 and actuate the first and third sets of visible indicators 120, 124, and at intervals during operation of the pump in the stand-by delivery mode, deactivate the third set of visible indicators 124 and actuate the audible alarm 104 and the second set of visible indicators 122.

In this fashion the infusion device 104 according to the embodiment of FIGS. 1 and 2 may provide information regarding the operation of the pump 102 that is consistent, and keeps the user aware of the continued operation of the pump in the stand-by mode where delivery of the fluid continues to the patient, although not at the same rate as the therapy delivery mode. That is, the first set of visible indicators 120 communicate that the pump 102 is in operation, while actuation of the third set of visible indicators 124 in combination with the first set of visible indicators 120 communicates that the pump 102 is in the standby delivery state, and not the therapy delivery state. In addition, the audible alarm 104 and the second set of visible indicators 122 can be used to communicate the transition between modes, or states, and can be used to, at intervals, communicate that the pump 102 is operating in the stand-by state. Consequently, the user is made aware of major changes in the operation of the infusion device (e.g., the change from therapy delivery mode to stand-by mode, and that the infusion device is continuing to operate stand-by mode and not therapy delivery mode).

Having thus discussed the infusion device 100 in general terms with reference to FIGS. 1 and 2, the infusion device 100 is now described in detail, and with reference to different pumps.

According to the embodiment of the infusion device 100 illustrated in FIGS. 1 and 2, the infusion device 100 has a housing 130 that may be attached to the pump 102, which as illustrated is a syringe pump. The housing 130 may be used to house or contain certain parts of the infusion device 100, or to have certain parts of the infusion device disposed thereon. For example, the audible alarm 104 and the pump controller 108 may be disposed in the housing 130, and the housing 130 may have a front face 132 on which the plurality of visible indicators 106 may be disposed.

According to one embodiment, the audible alarm 104 may be a speaker, such as a piezoelectric speaker, for example. Other forms of audio output devices may be used instead of the speaker as the audible alarm 104.

The plurality of visible indicators 106 may include one or more light emitting elements, such as light emitting diodes (LEDs) for example. As mentioned above, the plurality of visible indicators 106 may include at least three sets of visible indicators 120, 122, 124, as seen in FIG. 2. As illustrated, the three sets 120, 122, 124 are part of a two-dimensional array of visible indicators, which may be referred to as a linear array even though the elements of the array may not be in a straight line. Optionally, the sets 120, 122, 124 may be disposed in a three-dimensional array instead.

As is also illustrated in FIG. 2, each set 120, 122, 124 includes at least one pair of light emitting elements (e.g., LEDs). As illustrated, sets 120, 124 include a single pair of light emitting elements, while set 122 includes two pair of light emitting elements. As is also illustrated, each set is disposed with one of the light emitting elements of each pair on a different side (left or right) of the housing 130 of the infusion devices 100, which may facilitate the visibility of the visible indicators 106. According to other embodiments, each set 120, 122, 124 could include additional pairs of light emitting elements, or a set could even include a single light emitting element.

According to an embodiment, the light emitting elements (e.g., LEDs) of each of the sets 120, 122, 124 may display different colors when actuated (e.g., a first color, a second color, and a third color). For example, the light emitting elements of the first set 120 may display green light, the light emitting elements of the second set 122 may display red light, and the light emitting elements of the third set 124 may display yellow light. These colors may be a consequence of the element itself, or a colored filter applied to the element.

One or more of the sets 120, 122, 124 may be actuated so as to display a constant light, or may be actuated so as to flash on and off. The different lights and/or different actuation patterns may be used to differentiate the indicators of each of the sets 120, 122, 124 from the others, and/or the patterns may be used to draw attention to the information being communicated by the set 120, 122, 124. For example, the indicators of the second set 122 may be actuated to flash on and off to draw attention to the information being communicated thereby, i.e., that the pump 102 is changing modes or that the pump 102 has been operating in the stand-by mode for a period of time.

The infusion device 100 may include other output devices, and input devices as well. For example, the infusion device 100 may include one of more input devices in the form of buttons 134 disposed on the front face 132 of the housing 130, which buttons 134 may be electromechanical pushbutton switches, for example. The infusion device 100 may also include devices that function both as an input device and an output device. For example, a display device with associated touchscreen, defining a touch-sensitive display 136, may be disposed on the front face 132 of the housing 130, in addition to the buttons 134. The pump controller 108 may receive signals from the buttons 134 and the display 136 to allow a user to enter commands for the operation of the infusion device 100, and may send signals to the display 136 to communicate operational information regarding a process of an infusion operation being carried out using the infusion device 100.

The plurality of visible indicators 106 may be disposed adjacent to (touching or in close proximity to) the other input and/or output devices on the infusion device 100. As illustrated in FIGS. 1 and 2, the visible indicators 106 are disposed adjacent to the input devices (buttons 134, touchscreen 136) and other output devices (display 136). In this fashion, the visible indicators 106 communicate their information in such a way that it is not necessary for a user of the infusion device 100 to shift their view to different parts of the infusion device 100 during operation.

The input and output devices 104, 106, 134, 136 are coupled to the pump controller 108, which may include a microprocessor (which, in fact may include multiple physical and/or virtual processors). According to other embodiments, the controller 108 may include one or more electrical circuits designed to carry out the actions described herein. In fact, the controller 108 may include a microprocessor and other circuits or circuitry. In addition, the controller 108 may include one or more memories. The instructions by which the microprocessor is programmed may be stored on the one or more memories associated with the microprocessor, which memory/memories may include one or more tangible non-transitory computer readable memories, having computer executable instructions stored thereon, which when executed by the microprocessor, may cause the microprocessor to carry out one or more actions as described herein.

The infusion device 100 illustrated in FIG. 1 also includes a pump 102 in the form of a syringe pump. As such, the pump 102 includes a receptacle 140 in which the container 110, or a syringe, is disposed. The syringe 110 includes a cylindrical tube 142 and a plunger 144 movable within the cylindrical tube 142. At an end 146 of the cylindrical tube 142 opposite the plunger 144 is a delivery line 148 that extends from the cylindrical tube 142 towards a patient B and that may be part of an infusion set. The delivery line 148, which may be flexible tubing made of a PVC material for example, may be connected to the cylindrical tube 142 at an end 150 and to the patient B at an end 152 via a needle for providing intravenous access to the patient B for example.

As illustrated, the plunger 144 is in engagement with a pusher device 154 that is moved by an electromechanical drive, such as may be illustrated in US Pub. No. 2012/0215170 or US Pub. No. 2020/0030526, both of which are incorporated by reference herein in their entirety. In particular, the plunger 144 has a head 156 facing away from the cylindrical tube 142 and being in abutment with the pusher device 154 of the infusion device 100. In operation, the pusher device 154 is moved in an actuation direction A such that the plunger 144 is moved into the cylindrical tube 142 and a fluid (e.g., a medical fluid such as a medication) contained in the cylindrical tube 142 is delivered via the delivery line 148 towards the patient B.

As explained above, the plunger 144 may be moved into the cylindrical tube 142 such that a desired flow rate may be obtained, which rate or rates are programmed by a user prior to the start of the infusion operation. This has been referred to above as the therapy delivery mode or state. In addition, the plunger 144 may be moved into the cylindrical tube 142 such that a continuous flow rate is provided, which continuous flow rate maintains the vein of the patient B in an open state. This has been referred to above as the stand-by mode or state, or alternatively as a keep vein open (KVO) mode or state. In addition to these two modes, the infusion device 100 may have other modes of operation as well.

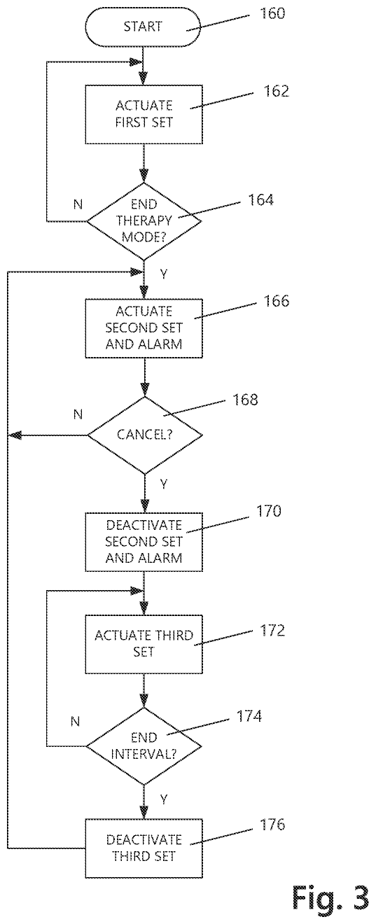

Having discussed the structure of the infusion device 100 with syringe pump 102 with reference to FIGS. 1 and 2, an embodiment of a method of operating the infusion device 100 with syringe pump 102, and in particular a method of operating the infusion device in conjunction with the user interface (including the audible alarm 104 and plurality of visible indicators 106) is discussed with reference to FIG. 3.

The method begins at block 160, when the pump 102 begins operation of the therapy delivery mode. As mentioned above, during the therapy delivery mode, the pump 102 delivers fluid from the container 110, for example to patient B, at a therapeutic rate that is programmed into the controller 108. During the therapy delivery mode, the pump controller 108 automatically actuates the first set of visible indicators 120 (e.g., green LEDs) at block 162. The controller 108 determines at block 164 if the end of the therapy delivery mode has been reached (e.g., if a particular delivery volume of fluid has been delivered from the container 110). If the end of the therapy delivery mode has not been reached, the method returns to block 162. If the therapy delivery mode has ended, the method continues to block 166.

As the therapy delivery mode has ended, the pump controller 108 changes from the therapy delivery mode to the stand-by deliver mode, which according to an embodiment of the present disclosure is a KVO mode. The pump controller also actuates the second set of visible indicators 122 and the audible alarm 104 at block 166. The second set of visible indicators 122 may be red LEDs, for example, and the pump controller may actuate the second set 122 to flash off and on (e.g., at 2 Hz) while the audible alarm 104 is also actuated.

The pump controller 108 then determines if a cancel command has been received at block 168. According to one embodiment, the infusion device 100 may include an input device, such as buttons 134, and when the input device is actuated, the pump controller may determine that the cancel command has been received. According to other embodiments, the cancel command may be generated with the passage of time. In any event, if the cancel command has not been received, then the method returns to block 166. If the cancel command has been received, then the method passes to block 170.

At block 170, the pump controller 108 deactivates the second set 122 and the audible alarm 104, at least for an interval of time. The interval may be periodic (e.g., every 1 to 5 minutes), and the interval may include a default value (e.g., every 2 minutes) that the user may change by programming the controller 108. According to other embodiments, the interval may not be periodic, and may vary depending on when the cancel command is received, for example.

With the pump 102 operating in the stand-by delivery mode (e.g., KVO mode), the pump controller 108 automatically actuates the third set of visible indicators 124 at block 172, such that the first and third sets of visible indicators 124 are now displayed. The third set 124 may include yellow LEDs, for example. The continuation of the display of the first set 120 communicates that the pump 102 is still delivering fluid, but the third set 122 communicates that the pump 102 is not delivering fluid at the programmed therapeutic rate, but at a KVO rate instead for example.

At block 174, the pump controller 108 determines if the interval since the last actuation of the second set of visible indicators 122 and the audible alarm 104 has elapsed. If it has not, then the method returns to block 172. If the interval has elapsed, then the pump controller deactivates the third set 124 at block 176, and the method returns to block 166, whereupon the second set 122 and the audible alarm 104 are actuated.

It will be recognized that the method of FIG. 3 is but one embodiment of a method of operating the infusion device 100 in conjunction with the user interface described herein. Other embodiments are possible within the scope of the present disclosure.

It will also be recognized that the method of FIG. 3 is not limited to an embodiment of the infusion device 100 including a syringe pump 102. For example, the pump may be a volumetric (peristaltic) pump, as is illustrated in FIG. 4.

Indeed, the infusion device 100′ illustrated in FIG. 4 includes a volumetric pump 102′, although the infusion device 100′ provides an outward appearance very similar to the infusion device 100 illustrated in FIGS. 1 and 2. For example, the infusion device 100′ includes a housing 130′ with a front face 132′ with buttons 134′ and a touch-sensitive display 136′ disposed on the front face 132′. The infusion device 100′ also includes a plurality of visible indicators 106′ disposed on the front face 132′ of the device 100′, adjacent to the buttons 134′ and the display 136′. The audible alarm and pump controller may be disposed within the housing 130′ as was the case in the embodiment of FIGS. 1 and 2.

Furthermore, the volumetric pump 102 may have a housing element, or door, 180 pivotable about an axis relative to the housing 130′, and behind the door 180 may be disposed a receptable in which a pump module of an infusion set may be received. The infusion set may be connected to a container in the form of a flexible bag, for example. In a closed state, such as is illustrated, the pump module is held in place within the housing 130′. Additional details of such a volumetric pump may be illustrated in PCT Pub. No. WO 2017/032498, which is incorporated by reference herein in its entirety, although according to other embodiments the volumetric pump may operate directly against a delivery line of the infusion set to move fluid through the infusion set without the inclusion of the pump module.

Thus, an improved method and system for operating an infusion device with an improved user interface has been described, which infusion device may incorporate a variety of pumps. The description provided above is intended for illustrative purposes, and is not intended to limit the scope of the disclosure to any particular method, system, apparatus, or device described herein.

1. An infusion device comprising:
a pump configured to be connected to a container and to deliver a fluid from the container during at least a therapy delivery mode and a stand-by delivery mode;
an audible alarm and a plurality of visible indicators, the plurality of visible indicators comprising at least a first set of visible indicators, a second set of visible indicators, and a third set of visible indicators; and
a pump controller coupled to the pump, the audible alarm, and the plurality of visible indicators,
the pump controller configured to operate the pump in the therapy delivery mode or the stand-by delivery mode, and to actuate the audible alarm and the plurality of visible indicators,
wherein the pump controller is configured to actuate the first set of visible indicators in the therapy delivery mode and the stand-by delivery mode, to actuate the second set of visible indicators and the audible alarm at an end of the therapy delivery mode and at intervals during the stand-by delivery mode, and to actuate the third set of visible indicators during the stand-by delivery mode when the second set of visible indicators is deactivated.

2. The infusion device according to claim 1, further comprising an input device coupled to the pump controller, wherein the pump controller is configured to deactivate the second set of visible indicators and the audible alarm when the input device is actuated and for an interval of time thereafter.

3. The infusion device according to claim 1, wherein the plurality of visible indicators comprises a plurality of light emitting diodes (LED), and the first set of visible indicators are green LEDs, the second set of visible indicators are red LEDs, and the third set of visible indicators are yellow LEDs.

4. The infusion device according to claim 1, wherein pump controller actuates the second set of visible indicators to flash on and off at the end of the therapy delivery mode and at intervals during the stand-by delivery mode.

5. The infusion device according to claim 1, wherein the plurality of visible indicators comprises a linear array of visible indicators including the first, second, and third sets of visible indicators.

6. The infusion device according to claim 5, further comprising a display screen, the linear array of visible indicators being disposed adjacent the display screen.

7. The infusion device according to claim 1, wherein the pump comprises a syringe pump or a volumetric pump.

8. The infusion device according to claim 7, wherein the container comprises a syringe if the pump comprises a syringe pump, and the container comprises a flexible bag coupled to an infusion set if the pump is a volumetric pump.

9. An infusion device comprising:
a pump configured to be connected to a container and to deliver a fluid from the container in at least a therapy delivery mode and a stand-by delivery mode;
an audible alarm and a plurality of visible indicators, the plurality of visible indicators comprising at least a first set of visible indicators, a second set of visible indicators, and a third set of visible indicators; and
a pump controller coupled to the pump, the audible alarm, and the plurality of visible indicators, and configured to:
operate the pump in a therapy delivery mode to deliver fluid from the container and actuate the first set of visible indicators,
end the therapy delivery mode and actuate the audible alarm and the first and second sets of visible indicators,
operate the pump in a stand-by delivery mode to deliver fluid from the container and actuate the first and third sets of visible indicators, and at intervals during operation of the pump in the stand-by delivery mode, deactivate the third set of visible indicators and actuate the audible alarm and the second set of visible indicators.

10. A method for operating an infusion device, the infusion device comprising a pump configured to be connected to a container, an audible alarm, and a plurality of visible indicators, the plurality of visible indicators comprising at least a first set of visible indicators, a second set of visible indicators, and a third set of visible indicators, the method comprising:
operating the pump in a therapy delivery mode to deliver fluid from the container during which the first set of visible indicators is automatically actuated;
ending the therapy delivery mode and automatically actuating the audible alarm and the first and second sets of visible indicators;
operating the pump in a stand-by delivery mode to deliver fluid from the container during which the first and third sets of visible indicators are automatically actuated;
at intervals during operation of the pump in the stand-by delivery mode, automatically deactivating the third set of visible indicators and automatically actuating the audible alarm and the second set of visible indicators.

11. The method according to claim 10, wherein the second set of visible indicators and the audible alarm are deactivated for a time interval upon actuation of an input device.

12. The method according to claim 10, wherein the second set of visible indicators to flash on and off when actuated.
